(19) United States
(12) Patent Application Publication
(10) Pub. No.: US 2018/0036216 A1
(43) Pub. Date: Feb. 8, 2018

(54) WIPES IMPREGNATED WITH NAIL POLISH REMOVER FORMULATION

(71) Applicant: PAKET CORPORATION, Chicago, IL (US)

(72) Inventor: Matthew W. Zoeller, Chicago, IL (US)

(73) Assignee: PAKET CORPORATION, Chicago, IL (US)

(21) Appl. No.: 15/227,891

(22) Filed: Aug. 3, 2016

(51) Int. Cl. A61K 8/37; A61K 8/42; B65D 83/08; A61K 8/34; A61K 8/49; A61K 8/86; A61Q 3/04; A61K 8/92

(52) U.S. Cl. CPC A61K 8/37; A61Q 3/04; A61K 8/42; A61K 8/922; A61K 8/34; A61K 8/4973; A61K 8/86; B65D 83/0805; A61K 2800/61; A61K 2800/5422; A61K 2800/5424; A61K 2800/596; A61K 2800/592; A61K 2800/87; A61K 2800/56

(57) ABSTRACT

A single-use wipe impregnated with a nail polish remover formulation comprising a hydroxyl-alkyl ester having formula (I):

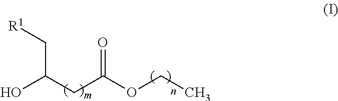

wherein R¹ is H, methyl, ethyl, or n-propyl; m is 1 to 3; and n is 2 to 6.

### FIELD OF THE INVENTION

The present invention is generally directed to nail polish remover formulations and more specifically to wipes impregnated with a nail polish remover formulation.

### BRIEF DESCRIPTION OF RELATED TECHNOLOGY

Conventional nail polish remover formulations contain one or more organic solvents such as acetone, ethyl acetate, or isopropyl alcohol, most typically, in amounts greater than 60 weight percent, based on the total weight of the nail polish remover formulation. Each of the foregoing organic solvents is a volatile organic compound (VOC) having a high vapor pressure at ordinary room temperature. These VOCs contribute to greenhouse gas accumulation and the depletion of our planet's ozone layer.

VOC's are organic chemical compounds that have sufficiently high vapor pressures under normal conditions to significantly vaporize and enter the atmosphere. 40 C.F.R. §51.100(s) defines VOC to include “any compound of carbon, excluding carbon monoxide, carbon dioxide, carbonic acid, metallic carbides or carbonates, and ammonium carbonate, which participates in atmospheric photochemical reactions.” There are numerous exempted solvents, however. For example, various halogenated solvents are excluded. Additionally, 40 C.F.R. §59.203(e) provides that fragrances which are incorporated into a consumer product up to a combined level of two weight-percent are exempted from the VOC regulations. 40 C.F.R. §59.203(f) also exempts organic compounds having a vapor pressure of less than 0.1 millimeters of mercury at 20° C., organic compounds having more than 12 carbon atoms, if the vapor pressure is unknown, or organic compounds having a melting point higher than 20° C. which do not sublime (i.e., compounds which do not change directly from a solid into a gas without melting), if the vapor pressure is unknown.

In any event, VOC's participate in reactions that result in tropospheric ozone formation (and smog). Further, high concentrations of VOC's have been associated with various health problems. Because of their negative effects on the environment and human well-being, such compounds are the subject of numerous regulations worldwide. Of course, such regulations apply to any compositions comprising solvents such as nail polish remover formulations. Thus, there is a need for cleaning compositions which do not contain VOC's in appreciable quantities.

Furthermore, the vapors generated from current nail polish remover products can create undesirable health effects for persons using them. For example, incidents of chronic exposure to acetone vapors have been reported.

Single-use wipes for cleaning skin portions are well-known. Such wipes are made of a nonwoven material and oftentimes include a lotion or aqueous solution to ease cleaning and to deliver skin benefits to the skin portion treated therewith. In this respect, for example, baby wipes are well-known. Single-use wipes impregnated with acetone solution for removing nail polish are also commercially available. To maintain the moistness of the wipe, these wipes are packaged in vapor impermeable packages. More specifically, such wipes are generally packaged either individually in a sealed package that can be torn open by the user, or in a dispenser containing a large number of wipes that may be dispensed through a dispenser opening in the dispenser.

### SUMMARY OF THE INVENTION

In one embodiment, the invention provides a single-use wipe comprising a nonwoven material impregnated with a nail polish remover formulation comprising a hydroxyl-alkyl ester having formula (I):

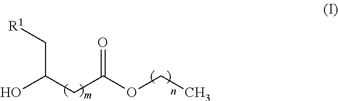

wherein R¹ is H, methyl, ethyl, or n-propyl; m is 1 to 3; and n is 2 to 6, wherein the hydroxyl-alkyl ester having formula (I) is the primary solvent of the nail polish remover formulation.

In another embodiment, the invention provides a sealed package comprising a sealed pouch comprising a composite structured film; a single-use wipe comprising a nonwoven material disposed in the sealed pouch film, the single-use wipe being impregnated with a nail polish remover formulation comprising a hydroxyl-alkyl ester having formula (I):

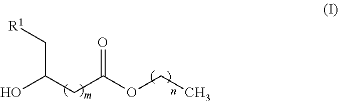

wherein R¹ is H, methyl, ethyl, or n-propyl; m is 1 to 3; and n is 2 to 6, wherein the hydroxyl-alkyl ester having formula (I) is the primary solvent of the nail polish remover formulation.

In yet another embodiment, the invention provides a dispenser comprising container having an opening; and a plurality of single-use wipes comprising a nonwoven material disposed in the container, the single-use wipes being impregnated with a nail polish remover formulation comprising a hydroxyl-alkyl ester having formula (I):

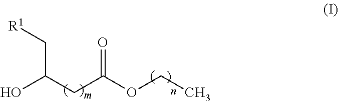

wherein R¹ is H, methyl, ethyl, or n-propyl; m is 1 to 3; and n is 2 to 6, wherein the hydroxyl-alkyl ester having formula (I) is the primary solvent of the nail polish remover formulation.

### BRIEF DESCRIPTION OF THE DRAWINGS

FIG. 1 is a cross-sectional view of a sealed pouch package containing a wipe in accordance with the present invention.

FIG. 2 is a cross-sectional view of a dispenser with a wipe being dispensed therefrom in accordance with the present invention.

### DETAILED DESCRIPTION

The present invention is particularly directed to single-use wipes impregnated with nail polish remover formulation comprising one or more hydroxyl-alkyl ester compounds according to formula (I):

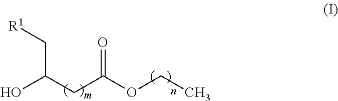

wherein R¹ is H, methyl, ethyl, or n-propyl; m is 1 to 3; and n is 2 to 6. In general, the nail polish remover formulation contains the hydroxyl-alkyl ester(s) according to formula (I) as the primary solvent. Advantageously, the hydroxyl-alkyl ester is a low vapor pressure volatile organic compound that does not contribute to ozone depletion, does not expose the user to noxious and unhealthy vapors, does not present undue health risks for the user's skin and eyes, and any residue after removal of nail polish rinses off readily with water rather than evaporating into the atmosphere as a non-fixed hydrocarbon.

As used herein, the term “primary solvent” refers to the solvent in the nail polish remover formulations which is contained in the greatest amount. Typically, the term refers to the nail polish remover formulation containing one or more of the hydroxyl-alkyl esters according to formula (I) in an amount greater than 50 weight percent (wt. %), for example, between 50 wt. % and 95 wt. %, between 60 wt. % and 90 wt. %, and/or between 65 wt. % and 85 wt. %, based on the total weight of the nail polish remover formulation.

The single-use wipes according to the invention may be safely used to remove nail polish (including but not limited to nail polish, lacquers, base coatings, and top coatings applied to human fingernails and toenails) from nails without exposing the user to undue chemical harm. In addition, to safely cleaning nail polish from finger nails, the single-use wipes according to the invention may be safely used to remove polyurethane-based paints, enamel paints, printing inks, and adhesives from hands.

As mentioned above, the nail polish remover formulations includes one or more compounds according to formula (I), typically as the predominant solvent. Specific suitable compounds according to formula (I) include but are not limited to propyl 3-hydroxybutanoate, butyl 3-hydroxybutanoate, pentyl 3-hydroxybutanoate, hexyl 3-hydroxybutanoate, heptyl 3-hydroxybutanoate, butyl 4-hydroxypentanoate, butyl 5-hydroxyhexanoate, butyl 3-hydroxypentanoate, butyl 3-hydroxyhexanoate, butyl 3-hydroxyheptanoate, and combinations of the foregoing. Butyl 3-hydroxybutanoate is a preferred hydroxyl-alkyl ester compound according to Formula (I) and is available under the OMNIA™ tradename (Eastman Chemical Company).

The nail polish remover formulation can be enhanced by incorporating other ingredients deemed safe for human skin contact. For example, the nail polish remover formulation can further comprise co-solvents, hydrotropes, surfactants, fragrances, removal aids, and combinations of the foregoing. Exemplary additional ingredients that may be included in the nail polish remover formulations include, but are not limited to, chemicals arising from the functional groups of esters, amides, ethers, acids and alcohols, provided that the additional ingredient(s) is miscible with the hydroxyl-alkyl ester primary solvent, is safe to use as a nail polish remover, and is cosmetically acceptable. Specific exemplary additional ingredients include amide co-solvents including but not limited to dimethyl lauramide, myristamide, and combinations thereof (which also advantageously function as hydrotopes in the nail polish remover formulations according to the invention); ester co-solvents including but not limited to C12-15 alkyl benzoates, isoamyl laurate, methyl acetate, and mixtures thereof; polyol cosolvents including but not limited to ethoxydiglycol; alcohol co-solvents including but not limited to benzyl alcohol; surfactants including but not limited to nonionic surfactants and anionic surfactants such as polyglyceryl-4 laurate, polygyceryl-3 oleate, polysorbate 60, sorbitan trioleate, ethoxylated C9-11 alcohols; naturally occurring essential oils (e.g., orange oil, peppermint oil) that are miscible with the nail polish remover formulation and can provide both enhanced solvency and aroma character; and combinations of the foregoing.

Even volatile organic compounds such as acetone, ethyl alcohol, and ethyl lactate may be included in the nail polish remover formulation to provide enhanced solvency benefits. When volatile organic compounds are included, the hydroxyl-alkyl ester according to formula (I) is typically the primary solvent such that any volatile organic compounds included in the nail polish remover formulations are not present in amounts sufficient to cause issues with chronic exposure and their contribution to greenhouse gas accumulation is greatly minimized. In this respect, the nail polish remover formulations of the invention generally contain less than 25 wt. % of volatile organic compounds, less than 20 wt. % volatile organic compounds, and/or less than 15 wt. % volatile organic compounds. In a preferred embodiment, the nail polish remover formulations are substantially free of volatile organic compounds. In the context of this invention, “substantially free of volatile organic compounds” means that the nail polish remover formulations contain less than about 5 wt. % of volatile organic compounds, based on the total weight of the formulation. More preferably, the nail polish remover formulations contain less than about 2 wt. %, and most preferably less than about 1 wt. % of volatile organic compounds.

The nail polish remover formulations may also include moisturizers such as aloe vera and propylene glycol. Water may also be present in the nail polish remover formulations according to the invention.

The single-use wipe according to the invention typically comprises a non-woven fibrous material. The non-woven fibrous material may be cellulosic-, polyester-, or polyolefin-based and may be manufactured “spun-bound”, i.e., by extruding, spun filaments onto a collecting belt in a uniform random manner followed by bonding the fibers. Of course, the non-woven fibrous material may also be manufactured as a staple non-woven or as a melt-blown non-woven as is well-known. Generally, the single-use wipe according to the invention comprises a single layer of the non-woven fibrous material, but of course, multi-layer constructions are possible. The single-use wipes can be impregnated with the nail polish remover formulation according to the discloser utilizing a spray nozzle or other suitable application method.

The single-use wipe according to the invention can be packaged individually in a sealed package that can be torn open by the user. Generally, the sealed package is a sealed pouch formed with a composite structured film. Linear low density polyethylene/Ethylene vinyl alcohol (LLDPE/EVOH) films are particularly suitable for manufacturing the sealed packages, as these films are substantially vapor-impermeable. Polyethylene terephthalate/low density polyethylene (PET/LDPE), PET/EVOH/LLDPE, and other films capable of providing barrier and sealant properties are also suitable for manufacturing the sealed packages. For example, PET faced laminated pouch films with EVOH, foil or metallocene barrier layers are also useful. Suitable films and laminated pouches are available from Bemis Company, Inc. and Avery Dennison.

Alternatively, the single-use wipe can be contained in a dispenser containing a large number of wipes that may be dispensed through a dispenser opening in the dispenser. In this aspect of the invention, the single-use wipes according to the invention can be in the form of a roll, with perforations between each individual wipe, the roll being disposed in a cylindrical dispenser. Of course, other configurations for dispensing the wipes are also contemplated. For example, the wipes can be vertically stacked, with perforations between each individual wipe facilitating removal of the wipes from the dispenser.

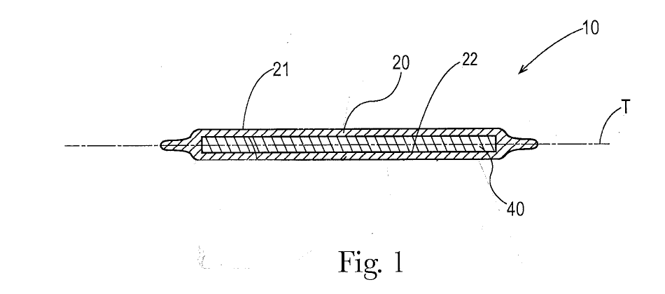

With reference now to the drawings, and particularly to FIG. 1, there is shown a cross sectional view of a pouch 10 including a wipe 40 contained therein along longitudinal axis T of the pouch 10. The pouch 40 is comprised of a composite structured film 20, the film including a first outer layer 21 and a second inner 22

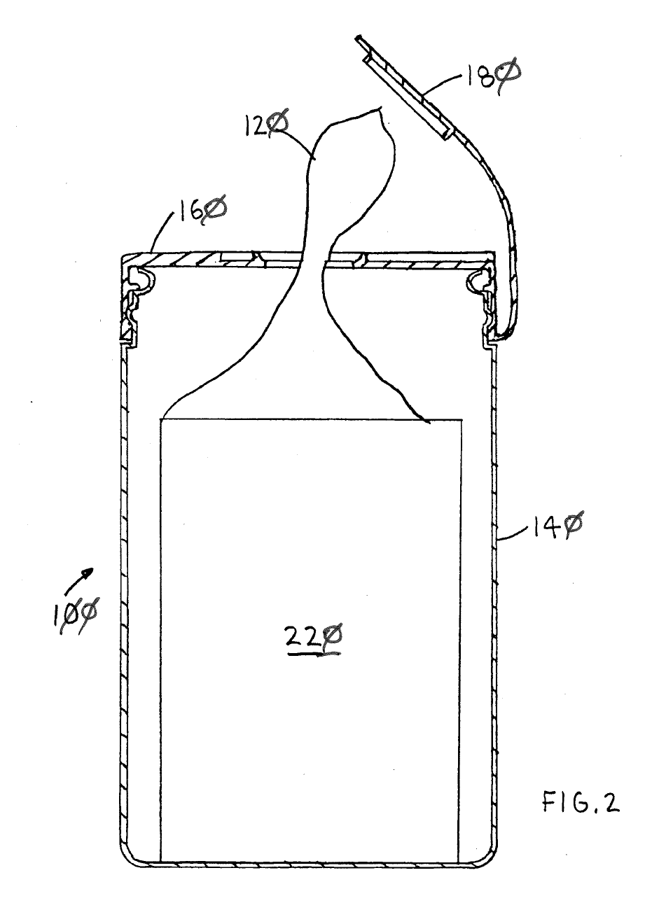

Referring to FIG. 2, a dispenser 100 includes a container 140, a dispenser cap 160 and a cover 180. The cover 180 preferably extends from a side of the dispenser cap 160. The cover 180 snaps on to a top of the dispenser cap 160 to prevent evaporation of the nail polish remover formulation from the wipes 120, 220. The dispenser cap 160 may be attached to a top of the container 140 with a snap fit. FIG. 2 illustrates the removal of at least one wipe 120 from the dispenser 100, while retaining the remaining wipes 220 in the container of the dispenser. Any suitable dispenser can be used.

To produce the nail polish remover formulation, the hydroxyl-alkyl ester according to formula (I) and any other components such as water, alcohol, moisturizer, fragrance, etc. are added to a phase tank and mixed until the mixture is homogenous, for example, for a period of five minutes.

The following examples are provided to illustrate the disclosure, but are not intended to limit the scope thereof.

### Example 1

Specific exemplary nail polish remover formulations according to the invention are provided in the tables below.

Nail Polish Remover Formulation 1

| Component | Content | Function |
|---|---|---|
| Butyl 3-hydroxybutanoate | 70.00 | principal ester solvent |
| Dimethyl lauramide/myristamide | 24.00 | amide co-solvent/hydrotrope |
| Isoamyl laurate | 3.00 | ester co-colvent |
| Aurantium dulcis (orange) oil | 3.00 | essential oil fragrance |
| Total | 100.00 | |

Nail Polish Remover Formulation 2

| Component | Content | Function |
|---|---|---|
| Butyl 3-hydroxybutanoate | 66.00 | principal solvent |
| Dimethyl lauramide/myristamide | 20.00 | hydrotrope/solvent |
| Ethoxydiglycol | 7.00 | polyol co-solvent |
| C12-15 alkyl benzoate | 5.00 | ester co-solvent |
| Polyglyceryl-4 laurate | 2.00 | surfactant/hydrotrope |
| Total | 100.00 | |

Nail Polish Remover Formulation 3

| Component | Content | Function |
|---|---|---|
| Butyl 3-hydroxybutanoate | 67.00 | principal solvent |
| Dimethyl lauramide/myristamide | 20.00 | hydrotrope/solvent |
| Benzyl Alcohol | 7.00 | alcohol co-solvent |
| Polysorbate 60 | 2.00 | nonionic surfactant |
| DEA Oleth-3 phosphate | 2.00 | anionic surfactant |
| Aurantium dulcis (orange) oil | 2.00 | essential oil fragrance |
| Total | 100.00 | |

Nail Polish Remover Formulation 4

| Component | Content | Function |
|---|---|---|
| Butyl 3-hydroxybutanoate | 66.00 | principal solvent |
| Dimethyl lauramide/myristamide | 22.00 | hydrotrope/solvent |
| Methyl acetate | 8.00 | ester co-solvent |
| Polygyceryl-3 oleate | 2.00 | nonionic surfactant/hydrotrope |
| Pepperintha mentha | 2.00 | essential oil fragrance |
| (peppermint) leaf oil | | |
| Total | 100.00 | |

Nail Polish Remover Formulation 5

| Component | Content | Function |
|---|---|---|
| Butyl 3-hydroxybutanoate | 70.00 | principal solvent |
| Dimethyl lauramide/myristamide | 15.00 | hydrotrope/solvent |
| Methyl acetate | 10.00 | ester co-solvent |
| Sorbitan trioleate | 2.00 | nonionic surfactant |
| C9-11 alcohols ethoxylated | 1.00 | nonionic surfactant |
| Pepperintha mentha (peppermint) | 2.00 | essential oil fragrance |
| leaf oil | | |
| Total | 100.00 | |

### Example 1

Exemplary test methods for confirming the efficacy of the nail polish remover formulations according to the invention are described herein. It is understood that adding the mechanical action of a wiping motion using a single-use wipe impregnated with nail polish remover formulation according to the invention will further expedite the removal of nail polish. Further, the following tests can be used to empirically compare the nail polish formulations according to the invention to conventional nail polish remover formulations.

Test Method (Detailed):
1. Clean, dry, number (e.g., etch) and weigh ten or more 1 inch by 3 inch glass microscope slides.
2. Apply one layer of polish base coat to one side of all slides. Allow 30 minutes to dry.
3. Apply one layer (evenly distributed) of a nail polish (colored) to all slides on side of slides including previously applied base coat. Allow 30 minutes to dry in fluorescent light or sunlight.
4. Apply top coat (evenly distributed) to all slides on side of slides including previously applied base coat and nail polish colorant coating. Allow 30 minutes to dry in fluorescent light or sunlight.
5. When dry, weigh each slide and record its gross and tare weight.
6. Fill five (5) 50 ml glass beakers to the 40 ml mark with a nail polish formulation according to the invention.
7. Set the timer and start timer when the (5) slides are placed into the beakers leaning against the inner wall with coated side facing up such that the treated side is immersed in the nail polish remover formulation.
8. Remove first slide at 20 seconds and the remainder at 20 second intervals for a total of 100 seconds immersion time for the fifth and final slide.
9. Rinse all slides with warm water for 30 seconds and gently wipe dry.
10. Weigh the slides and record the gross weight.
11. Record the net mass loss of nail polish coatings at each immersion interval.

Test Method (Empirical):
1. Prepare slides generally according to steps 1-5, above. Single layers of nail polish may be applied instead of applying each of a base coat, nail polish (colored), and top coat.
2. Prepare glass beakers according to step 6, above.
3. Immerse the dried, treated slide in the 40 ml of test solvent in a 50 ml beaker for 30 seconds.
4. Remove the slide and immediately wipe off nail polish layers using a disposable wipe (KIMWIPES®, Kimberly-Clark).
5. Rinse the slide off in warm water then allow to dry.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

1. A single-use wipe comprising:
a nonwoven material impregnated with a nail polish remover formulation comprising a hydroxyl-alkyl ester having formula (I): 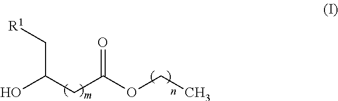
wherein R¹ is H, methyl, ethyl, or n-propyl; m is 1 to 3; and n is 2 to 6, wherein the hydroxyl-alkyl ester having formula (I) is the primary solvent of the nail polish remover formulation.

2. The single-use wipe according to claim 1, wherein the nail polish remover formulation comprises the hydroxyl-alkyl ester in an amount greater than 50 weight percent (wt. %).

3. The single-use wipe according to claim 1, wherein the hydroxyl-alkyl ester having formula (I) is selected from the group consisting of propyl 3-hydroxybutanoate, butyl 3-hydroxybutanoate, pentyl 3-hydroxybutanoate, hexyl 3-hydroxybutanoate, heptyl 3-hydroxybutanoate, butyl 4-hydroxypentanoate, butyl 5-hydroxyhexanoate, butyl 3-hydroxypentanoate, butyl 3-hydroxyhexanoate, butyl 3-hydroxyheptanoate, and combinations of the foregoing.

4. The single-use wipe according to claim 1, wherein the nail polish remover formulation further comprises one or more additional ingredient selected from the group consisting of co-solvents, hydrotropes, fragrances, removal aids, and combinations of the foregoing.

5. The single-use wipe according to claim 1, wherein the nail polish remover formulation further comprises one or more of acetone, ethyl alcohol, and ethyl acetate.

6. The single-use wipe according to claim 1, wherein the nail polish remover formulation contains less than 25 wt. % volatile organic compounds.

7. The single-use wipe according to claim 1, wherein the nail polish remover formulation is substantially free of volatile organic compounds.

8. The single-use wipe according to claim 1, wherein the nail polish remover formulation further comprises a moisturizer.

9. The single-use wipe according to claim 1, wherein the nail polish remover formulation further comprises water.

10. The single-use wipe according to claim 1, wherein the non-woven material comprises a cellulosic-based material, a polyester-based material, or a polyolefin-based material.

11. A sealed package comprising:
a sealed pouch comprising a composite structured film;
a single-use wipe comprising a nonwoven material disposed in the sealed pouch film, the single-use wipe being impregnated with a nail polish remover formulation comprising a hydroxyl-alkyl ester having formula (I): 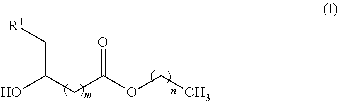
wherein R¹ is H, methyl, ethyl, or n-propyl; m is 1 to 3; and n is 2 to 6, wherein the hydroxyl-alkyl ester having formula (I) is the primary solvent of the nail polish remover formulation.

12. The sealed package according to claim 11, wherein the composite structure film comprises polyethylene terephthalate/low density polyethylene, linear low density polyethylene/ethylene vinyl alcohol, or polyethylene terephthalate/ethylene vinyl alcohol/linear low density polyethylene.

13. A dispenser comprising:
a container having an opening; and
a plurality of single-use wipes comprising a nonwoven material disposed in the container, the single-use wipes being impregnated with a nail polish remover formulation comprising a hydroxyl-alkyl ester having formula (I): 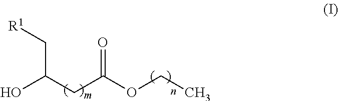
wherein R¹ is H, methyl, ethyl, or n-propyl; m is 1 to 3; and n is 2 to 6, wherein the hydroxyl-alkyl ester having formula (I) is the primary solvent of the nail polish remover formulation.
